(19) United States
(12) Patent Application Publication
(10) Pub. No.: US 2017/0281799 A1
(43) Pub. Date: Oct. 5, 2017

(54) SYSTEMS AND METHODS FOR SCREENING OTOTOXIC, OTOPROTECTIVE, AND OTOREGENERATIVE COMPOUNDS USING AQUATIC MODELS

(71) Applicant: ARIZONA BOARD OF REGENTS ON BEHALF OF THE UNIVERSITY OF ARIZONA, TUCSON, AZ (US)

(72) Inventors: Marvin SLEPIAN, Tucson, AZ (US); Abraham JACOB, Tucson, AZ (US); Maki NIIHORI, Tucson, AZ (US)

(21) Appl. No.: 15/504,758

(22) PCT Filed: Aug. 18, 2015
(86) PCT No.: PCT/US15/45634

§ 371 Date: Feb. 17, 2017

Related U.S. Application Data
(60) Provisional application No. 62/038,694, filed on Aug. 18, 2014.

(51) Int. Cl. A61K 49/00; G06F 19/00; G01N 33/00

(52) U.S. Cl. CPC A61K 49/0008; G01N 33/00; G06F 19/704; G01N 2001/002

(57) ABSTRACT

The present disclosure relates to devices, systems, and methods for screening compounds and materials for otoxicity and otoprotective activity. In particular, the present invention relates to a model organism (e.g., Danio rerio) and system for measuring behavior associated with hair cell loss.

The present application claims priority to U.S. Provisional Patent Application Ser. No. 62/038,694, filed Aug. 18, 2014, the disclosure of which is herein incorporated by reference in its entirety.

### STATEMENT REGARDING FEDERALLY SPONSORED RESEARCH OR DEVELOPMENT

This invention was made with government support under AJ-K08DC0094401A1 awarded by the National Institute of Deafness and Other Communication Disorders. The government has certain rights in the invention.

### FIELD OF THE INVENTION

The present disclosure relates to devices, systems, and methods for screening compounds and materials for otoxicity and otoprotective activity. In particular, the present invention relates to a model organism (e.g., Danio rerio) and system for measuring behavior associated with hair cell loss.

### BACKGROUND OF THE INVENTION

Hearing loss, defined as a partial or complete inability to perceive sound, is one of the most common human sensory disabilities. About 1:1000 children are born with severe to profound hearing loss, and 3-4:1000 develop hearing loss during childhood (Korver A M, et al., JAMA: the journal of the American Medical Association 2010; 304:1701-1708). Population-based studies in the United States suggest that by age 65, 42-47% of individuals are hearing-impaired in one or both ears (Cruickshanks K J, et al., American journal of epidemiology 1998; 148:879-886; Gates G A, et al., Ear and hearing 1990; 11:247-256; Moscicki E K, et al., Ear and hearing 1985; 6:184-190), and nearly all persons over age 80 have some degree of hearing loss (Lin F R, et al., Archives of internal medicine 2011; 171:1851-1852). Conservative estimates suggest that hearing impairment affects 32% of Americans aged 20-69 (Agrawal Y, et al., Arch Intern Med 2008; 168:1522-1530). Age related hearing loss is most common; however, tinnitus and hearing loss due to noise-exposure tops the list of war related healthcare costs, affecting over 1.5 million veterans. According to the DOD Hearing Center of Excellence, veterans receiving service-connected disability for tinnitus and hearing loss has increased 13-18% annually since 2000. The 2014 cost to the Department of Veterans Affairs is projected at $2.6 billion. Moreover, the personal and financial impact of recreational noise exposure is unquantifiable. Hearing loss has been shown to adversely impact communication, socialization, mood, physical functioning, and quality of life (Dalton D S, et al., The Gerontologist 2003; 43:661-668; Zwolan T A, et al., Ear and hearing 1996; 17:198-210). For decades it has been recognized that audition affects verbal and non-verbal cognitive functions (Ohta R J, et al., Journal of the American Geriatrics Society 1981; 29:476-478; Lindenberger U, et al., Psychology and aging 1994; 9:339-355), yet only recently has it been correlated with cognitive decline. Aging and noise exposure are the most common etiologies for hearing loss; however, due to inherent features, they are difficult to study using translational model systems. Ototoxicity is another known cause of sensorineural hearing loss, with cisplatin therapy a major contributor. The link between cisplatin treatment and ototoxicity in children and adults is well established (Coffin A B, et al., Zebrafish 2010; 7:3-11; Ou H C, et al., Drug Discov Today 2010; 15:265-271; Buck L, et al., J Pharmacol Tox Met 2012; 66:163-163) with nearly 55% of patients affected.

There are no FDA approved screens assaying the ototoxic components of drugs and ototoxic properties of most remain unknown. Like noise and aging, chemotherapy results in substantial inner ear damage to cochlear hair cells, resulting in permanent hearing loss for many cancer survivors (Berg A L, et al., Laryngoscope 1999; 109:1806-1814; Laurell G, et al., Laryngoscope 1990; 100:724-734; Rybak L P, et al., Hearing Res 2007; 226:157-167). Similarly environmental toxins, agents, chemical and materials as well can pose ototoxic risks. Moreover, inner ear hair cells are thought to undergo cell death from noise, ototoxicity, age, chemicals, and materials, etc. via similar pathways involving oxidative stress and free radical-mediated damage (Henderson D et al., Ear Hearing 2006; 27:1-19; Rybak L P, et al., Kidney Int 2007; 72:931-935; Hoshino T, et al., Biochemical and Biophysical Research Communications 2011; 415:94-98).

There are currently no FDA-approved pharmacological treatments specifically designated for the treatment of hearing loss; consequently, developing biological models for rapidly assaying drugs that might protect or potentially regenerate hearing is an urgent and unmet clinical need. Age related hearing loss is difficult to model, and research on noise-induced hearing loss typically requires use of higher order species like birds or rodents. The latter are expensive and low throughput biological systems for drug screening.

Research models leading to hearing preservation and regeneration would have profound implications.

### SUMMARY OF THE INVENTION

The present disclosure relates to devices, systems, and methods for screening compounds and materials for otoxicity and otoprotective activity. In particular, the present invention relates to a model organism (e.g., Danio rerio) and system for measuring behavior associated with hair cell loss.

Accordingly, in some embodiments, the present disclosure provides a method of screening compounds and/or materials for ototoxicity, comprising: a) contacting the compounds and/or materials with a fish, fish larvae, zooplankton, or other aquatic organism that exhibits altered behavior in response to anatomical loss of hair cells; and b) identifying compounds and/or materials that alter the behavior. The present disclosure is not limited to particular behaviors or fish. In some exemplary embodiments the behavior is rheotaxis and the fish is Danio rerio. In some embodiments, the compounds are pharmaceutical agents or candidate pharmaceutical agents, materials such a biomaterials, or yet unidentified, non-pharmaceutical biomaterials. In some embodiments, a plurality of doses of said compounds are tested on different fish or populations of fish (e.g., to calculate a dose-response relationship). In some embodiments, pluralities of distinct compounds are each contacted with different fish or populations of fish. In some embodiments, the method is a high throughput screening method. In some embodiments, the behavior is quantitated (e.g., on a numeric scale). In some embodiments, the quantitation is automated or manual.

Further embodiments provide a method of screening compounds for otoprotective or regenerative activity, comprising: a) contacting a fish that exhibits altered behavior in response to loss of hair cell mass with a ototoxic agent and a test compounds; and b) identifying test compounds that alter, treat, or prevent ototoxicity caused by the ototoxic agent by measuring changes in fish behavior. In some embodiments, the ototoxic agent is ototoxic agent is from cisplatin, carboplatin, aminoglycoside antibiotics, diuretics, salicylates, NSAIDs, quinine, solvents, or heavy metals.

Additional embodiments provide a system, comprising: a) a multichamber device comprising a plurality of wells, each well comprising one or more fish that exhibit altered behavior in response to exogenous agent exposure (e.g., in response to loss of hair cell mass); b) a fluid exchange system; and c) a detection of behavior and/or data analysis component. In some embodiments, the system comprises a microscope. In some embodiments, the fluid exchange system moves test compounds, ototoxic agents, or other reagents into the wells in an automated or semi-automated manner. In some embodiments, the wells are fully or partially translucent or transparent. In some embodiments, the system further comprises one or more test compounds (e.g., a candidate otoprotective agent, a candidate ototoxic agent, or a candidate hearing loss treatment/hearing loss regenerative agent). In some embodiments, the fish is Danio rerio. In some embodiments, the data analysis component calculates hair cell mass loss from the altered behavior. In some embodiments, the data analysis component comprises a camera (e.g., video and/or still camera), a computer processor and computer software. In some embodiments, devices are microfluidic devices that comprise one or more wells (e.g., 10 or more, 20 or more, 50 or more, 100 or more, etc), wherein each well comprises one or more fish that exhibit altered behavior in response to loss of hair cell mass.

Further embodiments provide compounds identified by the disclosed systems and methods (e.g., otoprotective agents).

Additional embodiments provide methods and uses of treating or preventing hearing loss, comprising: administering an agent that activates the NRF2 pathway (e.g., sulforaphane) to a subject at risk of hearing loss or having symptoms of hearing loss. In some embodiments, the agent (e.g., sulforaphane) alters, treats, or prevents ototoxicity caused by an ototoxic agent or has regenerative activity on hair cell loss.

Additional embodiments are described herein.

### DESCRIPTION OF THE DRAWINGS

FIG. 1 shows ventral neuromasts of posterior line P3-9.

FIG. 2 shows example images for anatomical scoring system. 0=no damage/normal staining; 1=mild damage/moderate staining; 2=severe damage/poor staining; 3=total neuromast/hair cell destruction/no staining.

FIG. 3 shows a schematic of an exemplary zebrafish Behavioral apparatus and image.

FIG. 4 shows an exemplary image acquisition sequence: 30 minutes of dark adaptation followed by 5 minutes of filming in total. 2 minutes with no flow, 1 minute flow adaptation, 2 minutes with flow.

FIG. 5 shows images of fish with rheotaxis and no rheotaxis.

FIG. 6 shows individual fish angles, with 0° representing the head oriented directly into flow.

FIG. 7 shows neuromast damage scores.

FIG. 8 shows representative pictures of neuromast protection/damage in each experimental condition.

FIG. 9 shows that damage score plots represent mean damage score (n=7; Cis 0 (control), n=3; Dex 5/Cis 0, n=5; Dex 5/Cis 1000)±S.D.

FIG. 10 shows rheotaxis index±S.D. across treatment groups.

FIG. 11A-C shows protective effects of sulforaphane on zebrafish hair cell function.

### DEFINITIONS

To facilitate an understanding of the present invention, a number of terms and phrases are defined below:

As used herein, the term “test material” or “material” refers to a compound, solid, liquid or gel substance, either homogeneous or heterogeneous, a mixture, suspension colloid, alloy or other blend In some embodiments, the test material is formulated physically so as to be dispersed through a fluid for testing and evaluation as described herein.

The terms “test compound” and “candidate compound” refer to any chemical entity, pharmaceutical, drug, and the like that is a candidate for use to treat or prevent a disease, illness, sickness, or disorder of bodily function (e.g., hearing loss) or otherwise augment, alter or modulate function, whether function is normal, abnormal or at a hypo- or hyper-level. Test compounds comprise both known and potential therapeutic compounds. A test compound can be determined to be therapeutic by screening using the screening methods of the present invention. Examples of test compounds include, but are not limited to, carbohydrates, monosaccharides, oligosaccharides, polysaccharides, amino acids, peptides, oligopeptides, polypeptides, proteins, nucleosides, nucleotides, oligonucleotides, polynucleotides, including DNA and DNA fragments, RNA and RNA fragments and the like, lipids, retinoids, steroids, drug, antibody, prodrug, glycopeptides, glycoproteins, proteoglycans and the like, and synthetic analogues or derivatives thereof, including peptidomimetics, small molecule organic compounds and the like, and mixtures thereof (e.g., that is a candidate for use to treat or prevent a disease, illness, sickness, or disorder of bodily function (e.g., hearing loss). Test compounds comprise both known and potential therapeutic compounds. A test compound can be determined to be therapeutic by screening using the screening methods of the present invention.

A “known therapeutic compound” refers to a therapeutic compound that has been shown (e.g., through animal trials or prior experience with administration to humans) to be effective in such treatment or prevention.

As used herein, the term “test compound library” refers to a mixture or collection of one or more compounds generated or obtained in any manner. Preferably, the library contains more than one compound or member. The test compound libraries employed in this invention may be prepared or obtained by any means including, but not limited to, combinatorial chemistry techniques, fermentation methods, plant and cellular extraction procedures and the like. Methods for making combinatorial libraries are well-known in the art (See, for example, E. R. Felder, Chimia 1994, 48, 512-541; Gallop et al., J. Med. Chem. 1994, 37, 1233-1251; R. A. Houghten, Trends Genet. 1993, 9, 235-239; Houghten et al., Nature 1991, 354, 84-86; Lam et al., Nature 1991, 354, 82-84; Carell et al., Chem. Biol. 1995, 3, 171-183; Madden et al., Perspectives in Drug Discovery and Design 2, 269-282; Cwirla et al., Biochemistry 1990, 87, 6378-6382; Brenner et al., Proc. Natl. Acad. Sci. USA 1992, 89, 5381-5383; Gordon et al., J. Med. Chem. 1994, 37, 1385-1401; Lebl et al., Biopolymers 1995, 37 177-198; and references cited therein. Each of these references is incorporated herein by reference in its entirety).

The term “synthetic small molecule organic compounds” refers to organic compounds generally having a molecular weight less than about 1000, preferably less than about 500, which are prepared by synthetic organic techniques, such as by combinatorial chemistry techniques.

As used herein the term “prodrug” refers to a pharmacologically inactive derivative of a parent “drug” molecule that requires biotransformation (e.g., either spontaneous, chemically mediated. or enzymatic) within the target physiological system to release, or to convert (e.g., enzymatically, mechanically, electromagnetically, etc.) the “prodrug” into the active “drug.” “Prodrugs” are designed to overcome problems associated with stability, toxicity, lack of specificity, or limited bioavailability. Exemplary “prodrugs” comprise an active “drug” molecule itself and a chemical masking group (e.g., a group that reversibly suppresses the activity of the “drug”). Some preferred “prodrugs” are variations or derivatives of compounds that have groups cleavable under metabolic conditions. Exemplary “prodrugs” become pharmaceutically active in vivo or in vitro when they undergo solvolysis under physiological conditions or undergo enzymatic degradation or other biochemical transformation (e.g., phosphorylation, hydrogenation, dehydrogenation, glycosylation, etc.). Prodrugs often offer advantages of solubility, tissue compatibility, or delayed release in the mammalian organism. (See e.g., Bundgard, Design of Prodrugs, pp. 7-9, 21-24, Elsevier, Amsterdam (1985); and Silverman, The Organic Chemistry of Drug Design and Drug Action, pp. 352-401, Academic Press, San Diego, Calif. (1992)). Common “prodrugs” include acid derivatives such as esters prepared by reaction of parent acids with a suitable alcohol (e.g., a lower alkanol), amides prepared by reaction of the parent acid compound with an amine (e.g., as described above), or basic groups reacted to form an acylated base derivative (e.g., a lower alkylamide).

As used herein, the term “sample” is used in its broadest sense. In one sense, it is meant to include a specimen or culture obtained from any source, as well as biological and environmental samples. Biological samples may be obtained from fish and/or animals (including humans) and encompass fluids, solids, tissues, and gases. Biological samples include blood products, such as plasma, serum and the like. Environmental samples include environmental material such as surface matter, soil, water, crystals and industrial samples. Such examples are not however to be construed as limiting the sample types applicable to the present invention.

As used herein, the term “effective amount” refers to the amount of a composition (e.g., a test compound) sufficient to effect beneficial or desired results (e.g., treat or prevent hearing loss). An effective amount can be administered in one or more administrations, applications or dosages and is not intended to be limited to a particular formulation or administration route.

As used herein, the term “administration” refers to the act of giving a drug, prodrug, or other agent (e.g., a test compound), or therapeutic treatment (e.g., compositions of the present invention) to a subject (e.g., a subject or in vivo, in vitro, or ex vivo cells, tissues, and organs). Exemplary routes of administration may be through the eyes (ophthalmic), skin (transdermal), by injection (e.g., intramuscularly, intravenously, subcutaneously, intratumorally, intraperitoneally, etc.) and the like. In some embodiments, agents are administered to the ear. Examples include, but are not limited to, oral, intratympanic application to the middle ear/mastoid, or direct introduction (vascular, microinjection, etc) to the inner ear.

As used herein, the term “co-administration” refers to the administration of at least two agent(s) (e.g., a test compound and one or more other agents) or therapies to a subject. In some embodiments, the co-administration of two or more agents or therapies is concurrent. In other embodiments, a first agent/therapy is administered prior to a second agent/therapy. Those of skill in the art understand that the formulations and/or routes of administration of the various agents or therapies used may vary. The appropriate dosage for co-administration can be readily determined by one skilled in the art. In some embodiments, when agents or therapies are co-administered, the respective agents or therapies are administered at lower dosages than appropriate for their administration alone. Thus, co-administration is especially desirable in embodiments where the co-administration of the agents or therapies lowers the requisite dosage of a potentially harmful (e.g., toxic) agent(s).

As used herein, the term “toxic” refers to any detrimental or harmful effects on a subject, a cell, or a tissue as compared to the same cell or tissue prior to the administration of the toxicant.

As used herein, the term “ototoxic” refers to any detrimental or harmful effect on a subject's hearing (e.g., by destruction of hair cells in the inner ear) and/or balance.

As used herein, the term “pharmaceutical composition” refers to the combination of an active agent (e.g., a test compound) with a carrier, inert or active, making the composition especially suitable for therapeutic use in vitro, in vivo or ex vivo.

The terms “pharmaceutically acceptable” or “pharmacologically acceptable,” as used herein, refer to composition that does not substantially produce adverse reactions, e.g., toxic, allergic, or immunological reactions, when administered to a subject.

As used herein, the term “pharmaceutically acceptable carrier” refers to any of the standard pharmaceutical carriers including, but not limited to, phosphate buffered saline solution, water, emulsions (e.g., such as an oil/water or water/oil emulsions), and various types of wetting agents, any and all solvents, dispersion media, coatings, sodium lauryl sulfate, isotonic and absorption delaying agents, disintrigrants (e.g., potato starch or sodium starch glycolate), and the like. The compositions also can include stabilizers and preservatives. For examples of carriers, stabilizers and adjuvants. (See e.g., Martin, Remington's Pharmaceutical Sciences, 15th Ed., Mack Publ. Co., Easton, Pa. (1975), incorporated herein by reference in its entirety). In some embodiments, pharmaceutically acceptable carriers are drug delivery vehicles (e.g., micro or nanoparticles, capsules—e.g. micro or nanocapsules, liposomes, slurries, hydrogels, salves, ointments, pastes and the like).

As used herein, the term “pharmaceutically acceptable salt” refers to any salt (e.g., obtained by reaction with an acid or a base) of a compound of the present invention that is physiologically tolerated in the target subject (e.g., a mammalian subject, and/or in vivo or ex vivo, cells, tissues, or organs). “Salts” of the compounds of the present invention may be derived from inorganic or organic acids and bases. Examples of acids include, but are not limited to, hydrochloric, hydrobromic, sulfuric, nitric, perchloric, fumaric, maleic, phosphoric, glycolic, lactic, salicylic, succinic, toluene-p-sulfonic, tartaric, acetic, citric, methanesulfonic, ethanesulfonic, formic, benzoic, malonic, sulfonic, naphthalene-2-sulfonic, benzenesulfonic acid, and the like. Other acids, such as oxalic, while not in themselves pharmaceutically acceptable, may be employed in the preparation of salts useful as intermediates in obtaining the compounds of the invention and their pharmaceutically acceptable acid addition salts.

Examples of bases include, but are not limited to, alkali metal (e.g., sodium) hydroxides, alkaline earth metal (e.g., magnesium) hydroxides, ammonia, and compounds of formula NW4+, wherein W is C1-4 alkyl, and the like.

Examples of salts include, but are not limited to: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, flucoheptanoate, glycerophosphate, hemisulfate, heptanoate, hexanoate, chloride, bromide, iodide, 2-hydroxyethanesulfonate, lactate, maleate, methanesulfonate, 2-naphthalenesulfonate, nicotinate, oxalate, palmoate, pectinate, persulfate, phenylpropionate, picrate, pivalate, propionate, succinate, tartrate, thiocyanate, tosylate, undecanoate, and the like. Other examples of salts include anions of the compounds of the present invention compounded with a suitable cation such as Na+, NH4+, and NW4+ (wherein W is a C1-4 alkyl group), and the like. For therapeutic use, salts of the compounds of the present invention are contemplated as being pharmaceutically acceptable. However, salts of acids and bases that are non-pharmaceutically acceptable may also find use, for example, in the preparation or purification of a pharmaceutically acceptable compound.

As used herein, the term “non-human animals” refers to all non-human animals including, but not limited to, vertebrates such as rodents, non-human primates, ovines, bovines, ruminants, lagomorphs, porcines, caprines, equines, canines, felines, ayes, pisces (e.g., bony fish and cartilaginous fish), etc.

As used herein, the term “purified” or “to purify” refers to the removal of components (e.g., contaminants) from a sample. For example, antibodies are purified by removal of contaminating non-immunoglobulin proteins; they are also purified by the removal of immunoglobulin that does not bind to the target molecule. The removal of non-immunoglobulin proteins and/or the removal of immunoglobulins that do not bind to the target molecule results in an increase in the percent of target-reactive immunoglobulins in the sample. In another example, recombinant polypeptides are expressed in bacterial host cells and the polypeptides are purified by the removal of host cell proteins; the percent of recombinant polypeptides is thereby increased in the sample.

As used herein, the term “cell culture” refers to any in vitro culture of cells. Included within this term are continuous cell lines (e.g., with an immortal phenotype), primary cell cultures, transformed cell lines, finite cell lines (e.g., non-transformed cells), and any other cell population maintained in vitro.

As used herein, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

### DETAILED DESCRIPTION OF THE INVENTION

The present disclosure relates to devices, systems, and methods for screening compounds for otoxicity and otoprotective activity. In particular, the present invention relates to a model organism (e.g., Danio rerio) and system for measuring behavior associated with hair cell loss.

The zebrafish (Danio rerio) has become a widely used model to study disease and in vivo drug development (Goessling W, et al., J Clin Oncol. 2007 Jun. 10; 25(17):2473-9; Peterson R T, et al., Methods Cell Biol. 2004; 76: 569-91). Zebrafish have neurosensory hair cells on their body surfaces that are aggregated into clumps called neuromasts, found at stereotypic positions along their lateral line. These hair cells are structurally similar to those within the human inner ear, making zebrafish an excellent model to study inner ear dysfunction (Coffin A B, et al., Zebrafish 2010; 7:3-11; Ou H C, et al., Drug Discov Today 2010; 15:265-271; Buck L, et al., J Pharmacol Tox Met 2012; 66:163-163; Ou H C, et al., Hearing Res 2007; 233:46-53). Additionally, due to their superficial location, zebrafish lateral line hair cells can be experimentally damaged by manipulating conditions in their water, (e.g. adding exogenous toxic agents).

Drug development research examining ototoxicity and related auditory effects, using fish, has focused on anatomic assessment of damage and rescue, using intrinsically low-throughput methodologies like time-intensive microscopy techniques. The work presented here focuses on developing and validating a “behavioral” zebrafish ototoxicity model for medium- to high-throughput analysis, measuring the efficacy of drugs protecting against, screening for agents that reverse hair cell damage, and screen compounds for ototoxicity. In normal fish, the induction of flow/current results in predictable “head-to current” swimming behavior called “rheotaxis” (Suli A, et al., Hair Cells. Plos One 2012; 7). During experiments conducted during the course of development of embodiments of the present disclosure, this principle was utilized to create a behavioral assay for ototoxicity, which evaluates damage to zebrafish hair cells and correlates well to measures of anatomical damage. This technique allows for higher throughput behavioral measures that serve as a surrogate for anatomical measures of hair cell damage.

Experiments described herein demonstrated a clear, quantifiable behavioral relationship between exogenous exposure of an ototoxic agent (e.g., cisplatin) and altered rheotactic swimming behavior in zebrafish. Further, a clear correlative relationship between decreased behavioral performance and anatomic evidence of neuromast damage was established, validating diminished rheotactic capability as a functional biomarker of ototoxicity. As many pharmacologic and biomaterial agents have the unfortunate burden of ototoxicity and no FDA-approved pharmacological agents presently exist which modulate or remediate hearing loss, developing biomarker systems to evaluate emerging agents or materials is needed.

The development of a high-throughput behavioral assay for ototoxicity utilizing zebrafish represents a major step towards biologically screening larger numbers of drugs, materials or constructs for the prevention/treatment of hearing loss. Such a screening system offers clear advantages over cell-based or organ culture techniques. Specifically, altered rheotactic behavior represents a physiologic and anatomic biomarker of ototoxicity, compared with simple in vitro cytological alterations. It is anticipated that with the utilization of such a system for drug discovery and development, accelerated recognition of ototoxic compounds is possible. Additionally this system allows identification of otoprotective and otoregenerative agents. This is significant since pharmaceutical companies are interested in the development of small molecule strategies for regeneration of damaged otologic tissues. Accordingly, embodiments of the present disclosure provide devices, systems and methods for screening test compounds for ototoxicity, otoprotective activity, treatment of hearing loss. Exemplary embodiments are described herein.

### I. Methods

Embodiments of the present disclosure provide methods of screening compounds (e.g., for research and clinical applications). In some embodiments, methods comprise the steps of contacting a fish or other aquatic species that exhibits behavioral changes in response to loss of hair cell mass with a test compound and/or ototoxic agent; and determining a measure of hair cell loss in response to the compound.

In some embodiments, assays are medium or high throughput (See e.g., below discussion of systems). In some embodiments, fish are dark adapted prior to contacting the fish with the test or ototoxic agent.

The present disclosure is not limited to a particular fish. Any fish or organism that has a hair cell mass or equivalent and exhibits a detectable behavioral change in response to loss of the hair cell mass can be utilized. In some embodiments, bony fish are used. In some embodiments, zooplankton such as jelly fish (e.g., Cnidaria) are utilized. In some embodiments, fish are tagged or labeled (e.g., via injection or engineering) to aid in detection.

The present disclosure is not limited to a particular behavior. Any behavior that correlates with hair cell loss is suitable for use in the systems and methods described herein. In some embodiments, the behavior is visually detectable (e.g., rheotaxis). In some embodiments, a numerical scale of behavior is utilized to quantitate behavioral changes that correlate to hair cell loss. In some embodiments, the numerical scale is used to determine ototoxic or otoprotective properties of test compounds (e.g., by comparison to the level in the absence of the test compound or in comparison to a cut-off value based on established population or individual norms).

In some embodiments, detection and/or quantitation of behavior changes is automated (e.g., via analysis of photos or videos of fish behavior). In some embodiments, automation is performed by computer processor and computer software. In some embodiments, the computer generates a report summarizes the quantitative results for multiple fish or populations of fish. In some embodiments, dose-response graphs or reports are generated.

The present disclosure is not limited to particular test compounds, test materials, or ototoxic agents. In some embodiments, the methods are used to screen compounds (e.g., candidate drugs or pharmaceutical agents) or test materials for the side effect of ototoxicity. In such embodiments, one or more doses of the test compounds are contacted with different fish or populations of fish and the behavior correlated with hair cell loss is quantitated. In some embodiments, a dose response curve is generated and used to determine a cut-off dosage for hearing loss. Such information is useful for screening libraries or lead compounds at the pre-clinical stage or post clinical stage.

In some embodiments, test compounds or materials are candidate otoprotective agents or agents for treating hearing loss (e.g., oto-regenerative agents). In such embodiments, fish are contacted with known ototoxic agents (e.g., cisplatin, carboplatin, aminoglycoside antibiotics, loop diuretics, quinine or heavy metals; see e.g., Ototoxic brochure by the League for the hard or hearing, Jul. 12, 2012; herein incorporated by reference in its entirety) prior to, after, or concurrently with the test compound. The effect of the test compound on hair cell loss is assayed by analyzing the associated behavior of the fish. In some embodiments, compounds that prevent or reverse hair cell loss in response to ototoxic agents are identified using the described methods.

In some embodiments, the test compound or agent is an external agent (e.g., electricity, noise, chemical compound, etc.).

The present invention is not limited by the type of test compound. In some embodiments, the test compound is one of a library of test compounds. The present invention is not limited by the type of test compound assayed. Indeed a variety of test compounds can be analyzed by the present invention including, but not limited to, any chemical entity, pharmaceutical, drug, known and potential therapeutic compounds, small molecule inhibitors, pharmaceuticals, a test compound from a combinatorial library (e.g., a biological library; peptoid library, spatially addressable parallel solid phase or solution phase library; synthetic library (e.g., using deconvolution or affinity chromatography selection), and the like. Examples of test compounds useful in the present invention include, but are not limited to, carbohydrates, monosaccharides, oligosaccharides, polysaccharides, amino acids, peptides, oligopeptides, polypeptides, proteins, nucleosides, nucleotides, oligonucleotides, polynucleotides, including DNA and DNA fragments, RNA and RNA fragments and the like, lipids, retinoids, steroids, glycopeptides, glycoproteins, proteoglycans and the like, and synthetic analogues or derivatives thereof, including peptidomimetics, small molecule organic compounds and the like, and mixtures thereof.

The test compounds of the present invention can be obtained using any of the numerous approaches in combinatorial library methods known in the art, including biological libraries; peptoid libraries (libraries of molecules having the functionalities of peptides, but with a novel, non-peptide backbone, which are resistant to enzymatic degradation but which nevertheless remain bioactive; See, e.g., Zuckennann et al., J. Med. Chem. 37: 2678-85 (1994)); spatially addressable parallel solid phase or solution phase libraries; synthetic library methods requiring deconvolution; the ‘one-bead one-compound’ library method; and synthetic library methods using affinity chromatography selection. The biological library and peptoid library approaches are preferred for use with peptide libraries, while the other four approaches are applicable to peptide, non-peptide oligomer or small molecule libraries of compounds (See, e.g., Lam (1997) Anticancer Drug Des. 12:145).

Examples of methods for the synthesis of molecular libraries can be found in the art, for example in: DeWitt et al., Proc. Natl. Acad. Sci. U.S.A. 90:6909 (1993); Erb et al., Proc. Nad. Acad. Sci. USA 91:11422 (1994); Zuckermann et al., J. Med. Chem. 37:2678 (1994); Cho et al., Science 261:1303 (1993); Carrell et al., Angew. Chem. Int. Ed. Engl. 33.2059 (1994); Carell et al., Angew. Chem. Int. Ed. Engl. 33:2061 (1994); and Gallop et al., J. Med. Chem. 37:1233 (1994).

Libraries of compounds may be presented in solution (See, e.g., Houghten, Biotechniques 13:412-421 (1992)), or on beads (See, e.g., Lam, Nature 354:82-84 (1991)), chips (See, e.g., Fodor, Nature 364:555-556 (1993)), bacteria or spores (See, e.g., U.S. Pat. No. 5,223,409; herein incorporated by reference), plasmids (See, e.g., Cull et al., Proc. Nad. Acad. Sci. USA 89:18651869 (1992)) or on phage (See, e.g., Scott and Smith, Science 249:386-390 (1990); Devlin Science 249:404-406 (1990); Cwirla et al., Proc. Natl. Acad. Sci. 87:6378-6382 (1990); Felici, J. Mol. Biol. 222:301 (1991)).

### II. Devices and Systems

Embodiments of the present disclosure provide devices and systems for research and screening applications (e.g., screening compounds). In some embodiments, devices are channel or microfluidic devices that comprise one or more wells (e.g., 10 or more, 20 or more, 50 or more, 100 or more, etc), wherein each well comprises one or more fish that exhibit altered behavior in response to loss of hair cell mass. In some embodiments, devices comprise 32 lanes. In some embodiments, each lane comprises a dedicated camera (e.g. video or still camera).

In some embodiments, the wells are translucent or transparent. In some embodiments, systems comprise a fluid exchange component for transferring test compounds or ototoxic agents to the fish. In some embodiments, systems further comprise a data analysis component (e.g., comprising one or more of a microscope, a camera (e.g., video or still camera), computer, computer software, etc.). In some embodiments, still images are obtained every 10 seconds or less (e.g., every 5, 3, 1, second or less). In some embodiments, video is obtained and the full video is analyzed. In some embodiments, an automated tracking system that analyzes video footage in real time is utilized.

In some embodiments, data analysis is automated or manual. In some embodiments, the software provides reports such as, for example, dose response reports or ototoxicity reports.

### III. Therapeutic Applications

Embodiments of the present disclosure provide agents (e.g., small molecule drugs, protein drugs, nucleic acids, mimetics, etc.) identified by the above-described screening methods. In some embodiments, agents treat or prevent hearing loss (e.g., by protecting hair cells or promoting hair cell growth, although the present disclosure is not limited to a particular mechanism). In some embodiments, the agent is sulforaphane or a derivative or mimetic thereof.

In some embodiments, agents are provided in pharmaceutical compositions. Contemplated formulations include those suitable oral, rectal, nasal, topical (including transdermal, buccal and sublingual), vaginal, parenteral (including subcutaneous, intramuscular, intravenous and intradermal) and pulmonary administration. In some embodiments, formulations are conveniently presented in unit dosage form and are prepared by any method known in the art of pharmacy. Such methods include the step of bringing into association the active ingredient with the carrier which constitutes one or more accessory ingredients. In general, the formulations are prepared by uniformly and intimately bringing into association (e.g., mixing) the active ingredient with liquid carriers or finely divided solid carriers or both, and then if necessary shaping the product.

Formulations of the present invention suitable for oral administration may be presented as discrete units such as capsules, cachets or tablets, wherein each preferably contains a predetermined amount of the active ingredient; as a powder or granules; as a solution or suspension in an aqueous or non-aqueous liquid; or as an oil-in-water liquid emulsion or a water-in-oil liquid emulsion. In some embodiments, the active ingredient is presented as a bolus, electuary, or paste, etc.

In some embodiments, tablets comprise at least one active ingredient and optionally one or more accessory agents/carriers are made by compressing or molding the respective agents. In some embodiments, compressed tablets are prepared by compressing in a suitable machine the active ingredient in a free-flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface-active or dispersing agent. Molded tablets are made by molding in a suitable machine a mixture of the powdered compound (e.g., active ingredient) moistened with an inert liquid diluent. Tablets may optionally be coated or scored and may be formulated so as to provide slow or controlled release of the active ingredient therein using, for example, hydroxypropylmethyl cellulose in varying proportions to provide the desired release profile. Tablets may optionally be provided with an enteric coating, to provide release in parts of the gut other than the stomach.

Formulations suitable for topical administration in the mouth include lozenges comprising the active ingredient in a flavored basis, usually sucrose and acacia or tragacanth; pastilles comprising the active ingredient in an inert basis such as gelatin and glycerin, or sucrose and acacia; and mouthwashes comprising the active ingredient in a suitable liquid carrier. Pharmaceutical compositions for topical administration according to the present invention are optionally formulated as ointments, creams, suspensions, lotions, powders, solutions, pastes, gels, sprays, aerosols or oils. In alternatively embodiments, topical formulations comprise patches or dressings such as a bandage or adhesive plasters impregnated with active ingredient(s), and optionally one or more excipients or diluents. In some embodiments, the topical formulations include a compound(s) that enhances absorption or penetration of the active agent(s) through the skin or other affected areas. Examples of such dermal penetration enhancers include dimethylsulfoxide (DMSO) and related analogues.

If desired, the aqueous phase of a cream base includes, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof.

In some embodiments, oily phase emulsions of this invention are constituted from known ingredients in a known manner. This phase typically comprises an lone emulsifier (otherwise known as an emulgent), it is also desirable in some embodiments for this phase to further comprises a mixture of at least one emulsifier with a fat or an oil or with both a fat and an oil. Preferably, a hydrophilic emulsifier is included together with a lipophilic emulsifier so as to act as a stabilizer. It some embodiments it is also preferable to include both an oil and a fat. Together, the emulsifier(s) with or without stabilizer(s) make up the so-called emulsifying wax, and the wax together with the oil and/or fat make up the so-called emulsifying ointment base which forms the oily dispersed phase of the cream formulations.

Emulgents and emulsion stabilizers suitable for use in the formulation of the present invention include Tween 60, Span 80, cetostearyl alcohol, myristyl alcohol, glyceryl monostearate and sodium lauryl sulfate.

The choice of suitable oils or fats for the formulation is based on achieving the desired properties (e.g., cosmetic properties), since the solubility of the active compound/agent in most oils likely to be used in pharmaceutical emulsion formulations is very low. Thus creams should preferably be a non-greasy, non-staining and washable products with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for topical administration to the eye also include eye drops wherein the active ingredient is dissolved or suspended in a suitable carrier, especially an aqueous solvent for the agent.

Formulations for rectal administration may be presented as a suppository with suitable base comprising, for example, cocoa butter or a salicylate.

Formulations suitable for vaginal administration may be presented as pessaries, creams, gels, pastes, foams or spray formulations containing in addition to the agent, such carriers as are known in the art to be appropriate.

Formulations suitable for nasal administration, wherein the carrier is a solid, include coarse powders having a particle size, for example, in the range of about 20 to about 500 microns which are administered in the manner in which snuff is taken, i.e., by rapid inhalation (e.g., forced) through the nasal passage from a container of the powder held close up to the nose. Other suitable formulations wherein the carrier is a liquid for administration include, but are not limited to, nasal sprays, drops, or aerosols by nebulizer, an include aqueous or oily solutions of the agents.

Formulations suitable for parenteral administration include aqueous and non-aqueous isotonic sterile injection solutions which may contain antioxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. In some embodiments, the formulations are presented/formulated in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described. Preferred unit dosage formulations are those containing a daily dose or unit, daily subdose, as herein above-recited, or an appropriate fraction thereof, of an agent.

It should be understood that in addition to the ingredients particularly mentioned above, the formulations of this invention may include other agents conventional in the art having regard to the type of formulation in question, for example, those suitable for oral administration may include such further agents as sweeteners, thickeners and flavoring agents. It also is intended that the agents, compositions and methods of this invention be combined with other suitable compositions and therapies. Still other formulations optionally include food additives (suitable sweeteners, flavorings, colorings, etc.), phytonutrients (e.g., flax seed oil), minerals (e.g., Ca, Fe, K, etc.), vitamins, and other acceptable compositions (e.g., conjugated linoelic acid), extenders, and stabilizers, etc.

In some embodiments, compounds of embodiments of the present invention are coated on medical devices (e.g., including but not limited to, pacemakers, indwelling catheters, implants, joint replacements, bone repair devices and the like).

### C. Exemplary Administration Routes and Dosing Considerations

Various delivery systems are known and can be used to administer a therapeutic agent (e.g., S. aureus or S. epidermidis biofilm inhibitor) of the present invention, e.g., encapsulation in liposomes, microparticles, microcapsules, receptor-mediated endocytosis, and the like. Methods of delivery include, but are not limited to, intra-arterial, intramuscular, intravenous, intranasal, and oral routes. In specific embodiments, it may be desirable to administer the pharmaceutical compositions of the invention locally to the area in need of treatment; this may be achieved by, for example, and not by way of limitation, local infusion during surgery, injection, or by means of a catheter.

The agents identified herein as effective for their intended purpose can be administered to subjects or individuals susceptible to or at risk of developing S. aureus and/or S. epidermidis biofilm infection and conditions correlated with this. When the agent is administered to a subject such as a mouse, a rat or a human patient, the agent can be added to a pharmaceutically acceptable carrier and systemically or topically administered to the subject. To determine patients that can be beneficially treated, a tissue sample is removed from the patient and the cells are assayed for sensitivity to the agent.

In some embodiments, in vivo administration is effected in one dose, continuously or intermittently throughout the course of treatment. Methods of determining the most effective means and dosage of administration are well known to those of skill in the art and vary with the composition used for therapy, the purpose of the therapy, the target cell being treated, and the subject being treated. Single or multiple administrations are carried out with the dose level and pattern being selected by the treating physician.

Suitable dosage formulations and methods of administering the agents are readily determined by those of skill in the art. Preferably, the compounds are administered at about 0.01 mg/kg to about 200 mg/kg, more preferably at about 0.1 mg/kg to about 100 mg/kg, even more preferably at about 0.5 mg/kg to about 50 mg/kg. When the compounds described herein are co-administered with another agent (e.g., as sensitizing agents), the effective amount may be less than when the agent is used alone.

The pharmaceutical compositions can be administered orally, intranasally, parenterally or by inhalation therapy, and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present invention, the pharmaceutical compositions can also contain other pharmaceutically active compounds or a plurality of compounds of the invention.

More particularly, an agent of the present invention also referred to herein as the active ingredient, may be administered for therapy by any suitable route including, but not limited to, oral, rectal, nasal, topical (including, but not limited to, transdermal, aerosol, buccal and sublingual), vaginal, parental (including, but not limited to, subcutaneous, intramuscular, intravenous and intradermal) and pulmonary. It is also appreciated that the preferred route varies with the condition and age of the recipient, and the disease being treated.

Ideally, the agent should be administered to achieve peak concentrations of the active compound at sites of disease. This may be achieved, for example, by the intravenous injection of the agent, optionally in saline, or orally administered, for example, as a tablet, capsule or syrup containing the active ingredient.

Desirable blood levels of the agent may be maintained by a continuous infusion to provide a therapeutic amount of the active ingredient within disease tissue. The use of operative combinations is contemplated to provide therapeutic combinations requiring a lower total dosage of each component agent than may be required when each individual therapeutic compound or drug is used alone, thereby reducing adverse effects.

### EXPERIMENTAL

The following examples are provided in order to demonstrate and further illustrate certain preferred embodiments and aspects of the present invention and are not to be construed as limiting the scope thereof.

### Example 1

### Methods

### Zebrafish Care and Breeding

Zebrafish embryos were obtained from parings of AB Wild-type adult fish and maintained in E2 embryo medium (15.0 mM NaCl, 0.5 mM KCl, 1.0 mM MgSO₄, 0.15 mM KH₂PO₄, 0.05 mM Na₂HPO₄, 1.0 mM CaCl₂, 0.7 mM NaHCO₃) (Westerfeld M. The Zebrafish Book. A guide for the laboratory use of Zebrafish (Danio rerio). 5th ed. University of Oregon Press. Eugene; 2007. Chapter 3, Embryonic and Larval Culture; p.3.1-3.104) at a density of 50 embryos per 90 mm petri dish. All zebrafish were housed in a Zebrafish Aquatic Housing System (Aquaneering, Inc., San Diego, Calif., U.S.A.) located in the University's animal care facility. Environmental conditions: Light/Dark cycle: 14 h/10 h. Water temperature was controlled at 28.5±0.5° C. The University's Institutional Animal Care and Use Committee approved all animal procedures.

### Anatomical Assay

Following the drug exposure (see below for detailed methods), larvae were fluorescently stained/labeled in embryo medium containing 6 μM YO-PRO1 (Invitrogen; Y3603) for 20 min. YO-PRO1 is a common vital dye used for staining viable lateral line hair cells in zebrafish (Coffin A B, et al., Zebrafish. 2010 March; 7(1):3-11; Ou H C, et al., Drug Discov Today. 2010 April; 15(7-8):265-71; Ou H C, et al., Hear Res. 2007 November; 233(1-2):46-53; Santos F, et al., Hear Res. 2006 March; 213(1-2):25-33). Concentration and duration were optimized for this purpose. After labeling, the larvae were rinsed three times in embryo media and then anesthetized using 0.5 mM of MS-222 (Tricaine Methanesulfonate, Western Chemical Inc.) (Buck L M, et al., Hear Res. 2012 February; 284(1-2):67-81). The larvae were fixed in 2% paraformaldehyde for 1 hour at room temperature and stored in PBS overnight at 4° C. Fixed larvae were then mounted in 6% methylcellulose (Sigma; M7140) in 0.2 mm silicone chambers and covered with No. 1.5 cover glass (Warner Instruments; 64-0718) for confocal microscopy. Larvae were observed under a confocal microscope (Leica SP5) using 20× dry and 40× oil immersion lenses.

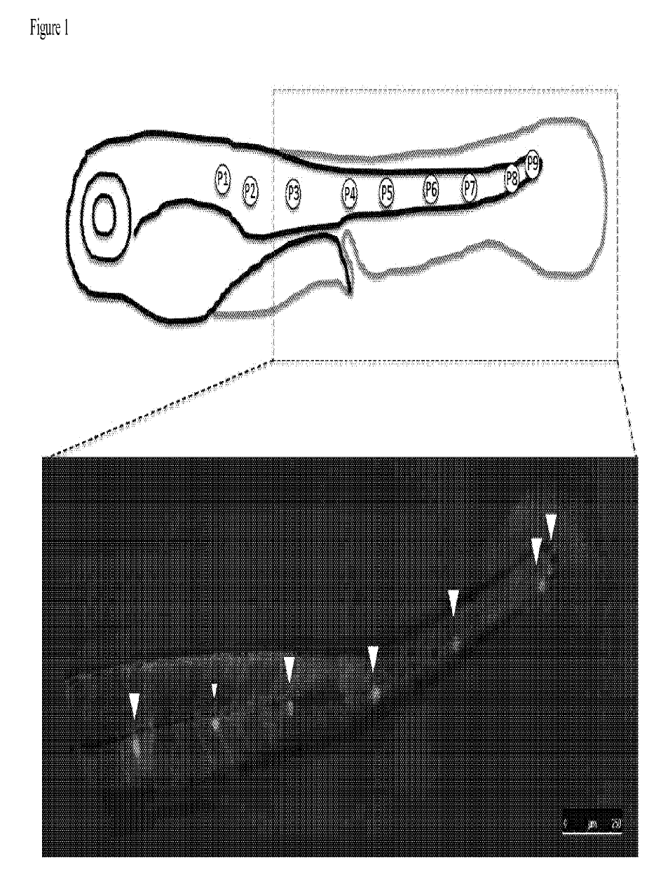

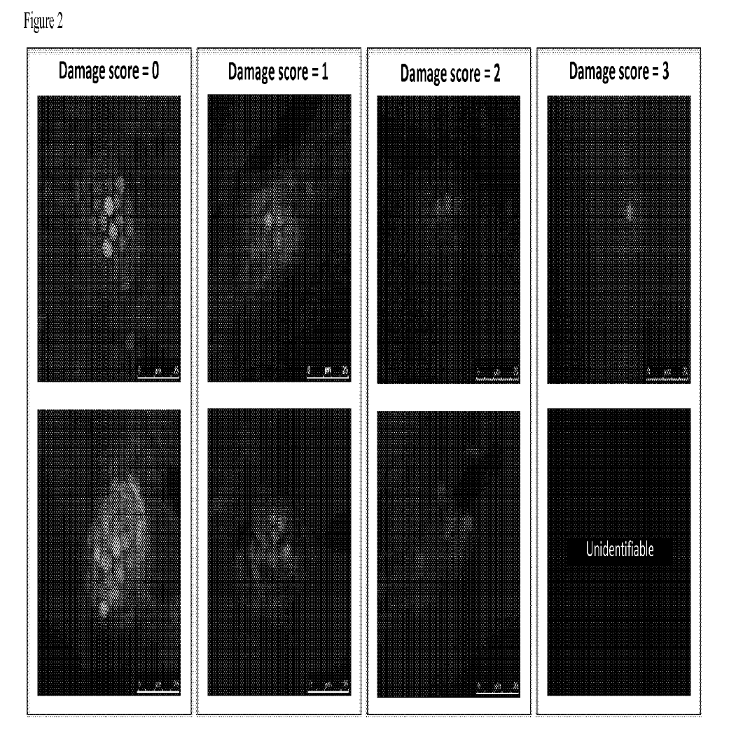

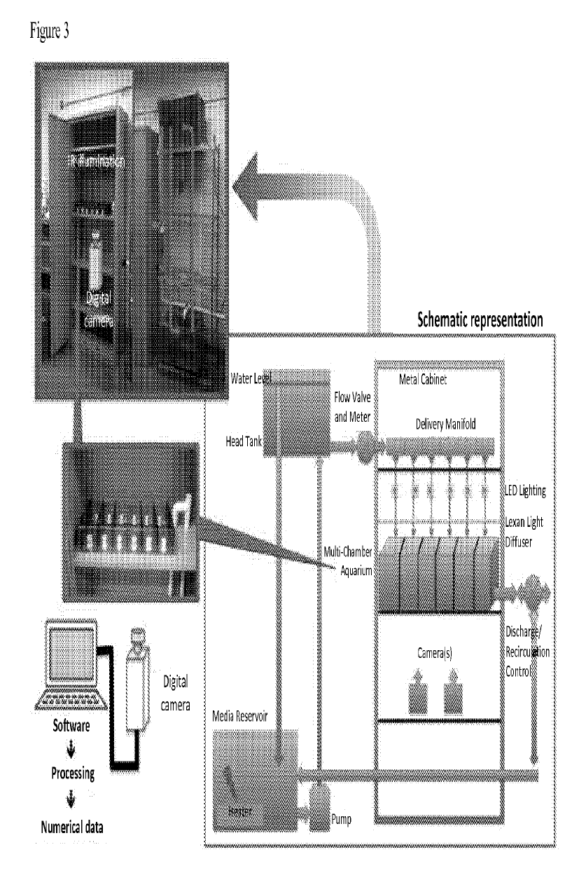

Treatment groups consisted of 3-7 fish. Seven ventral neuromasts of the posterior lateral line (P3, P4, P5, P6, P7, P8, and P9) were selected for determination of the “neuromast damage score” per fish (FIG. 1). Each neuromast was scored from 0 to 3. A score of 0 represents no damage with normal staining; 1 represents mild damage with moderate staining; 2 represents severe damage with poor staining; 3 represents total hair cell/neuromast destruction with no staining (FIG. 2). Seven scores per fish were then added to generate the total “aggregate neuromast damage score.” The lowest possible score for an individual fish is 0, representing no damage, with the highest possible score for a fish being 21, representing complete damage. Separate individuals (scorer A, B and C) were trained in the counting technique and “neuromast damage scores” were generated independently with blinding of experimental conditions to insure unbiased assessment. The mean score of the three evaluators was used to determine the final “neuromast damage score.” Inter-scorer variation was validated by using the Pearson's correlation coefficient.

### Behavioral Assay—Swimming Test Apparatus

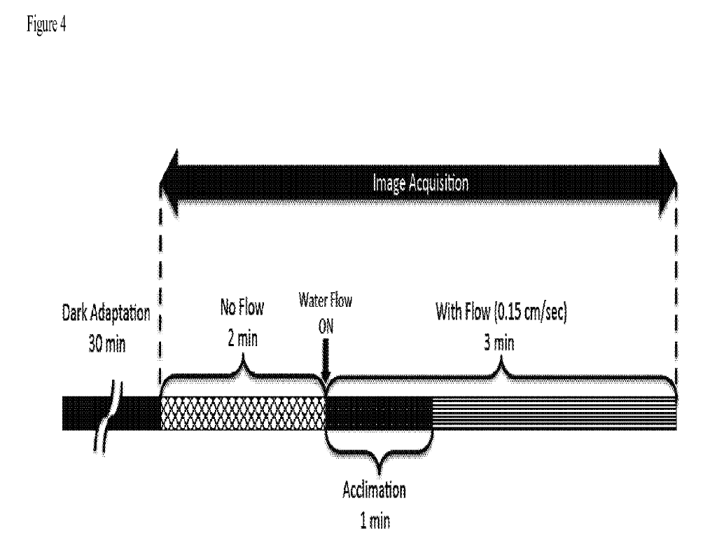

The custom-built behavioral apparatus consists of 6 experimental lanes with laminar flow velocity controlled at 0.15 cm/sec, allowing the simultaneous testing of up to 6 differing experimental conditions. Each lane measures 10.2 cm in height×9.9 cm in width×25.4 cm in length. Lanes are partitioned with metal mesh dividers to contain fish in one central area, measuring 5.0 cm length×9.9 cm width, allowing for image capture (FIG. 3). E2 medium flows from a header tank through the experimental lanes to a media reserve tank and is then recycled via pump back to the header tank. The pump is used to overdrive a header standpipe, resulting in constant pressure that is split evenly between the lanes, controlling the flow. A heater located in the reserve media tank maintains temperature at 28±0.5° C. Total system volume capacity (experimental apparatus+header and reserve tanks) is approximately 210 L, while operating volume is 152 L. The flow is calibrated as follows: lane width (9.9 cm)×total number of lanes (6)×operational water height (5.8 cm)×desired flow (0.15 cm/sec)=flow rate of 51.7 cubic centimeters/seconds. Water traveling from experimental lanes to the reserve media tank is received in a 1000 mL beaker and the rate of water flow is adjusted until it takes 19.3 seconds to fill the beaker to 1000 mL. Flow speed is calibrated before every experiment. With the system calibrated, flow may then be turned on or off via a separate valve, without any further adjustments.

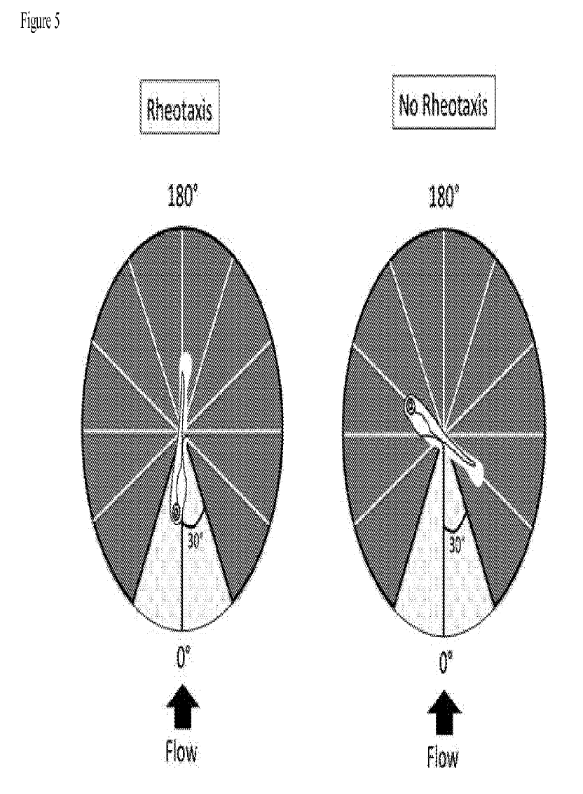

The experimental system is housed in a cabinet creating a condition free of ambient light, thus negating any influence of visual input on zebrafish swimming behavior. Infrared light provides lighting from which images were acquired. Video capture is accomplished with under-mounted Samsung SCB-200 high-resolution video cameras (FIG. 3). In conducting the assay, five-day post fertilization (5 dpf) zebrafish are treated with several different experimental conditions (see below for detailed methods), rinsed three times in embryo medium, transferred to the experimental lanes, and the cabinet is then closed for 30 minutes to allow for dark adaptation. Image acquisition begins with 2 minutes of filming under no-flow conditions, next the water flow is turned on and the fish are allowed 1 minute to acclimate to the flow and then 2 minutes of flow behavior is recorded (FIG. 4).

### Behavioral Assay—Data Generation

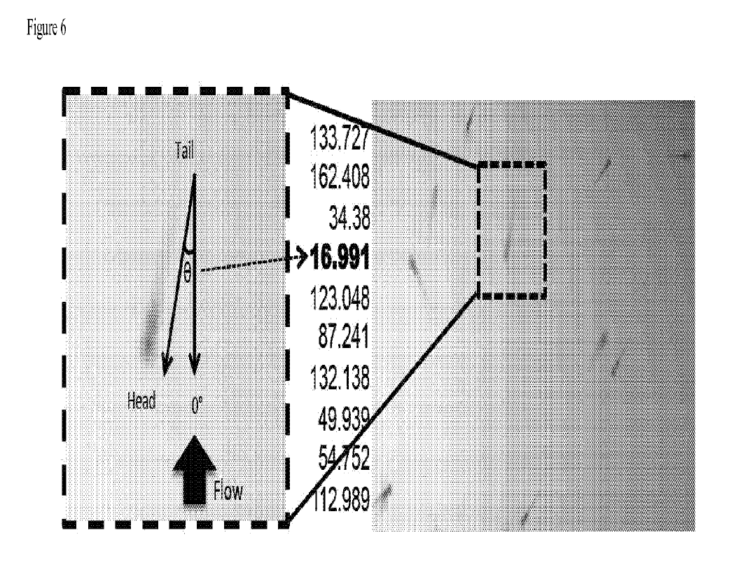

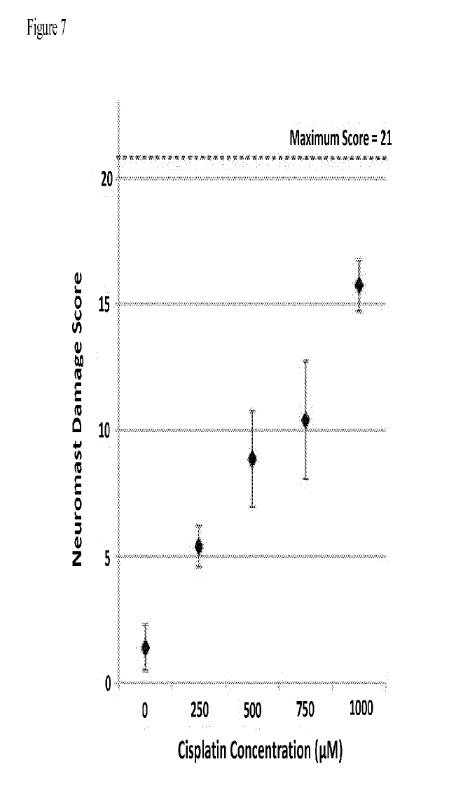

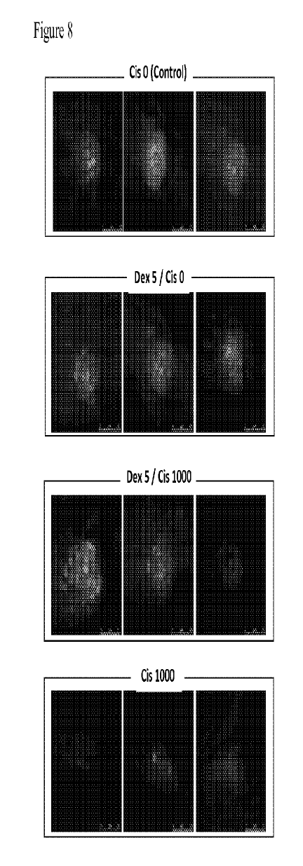

Rheotaxis was the zebrafish behavior under observation and a decrease in the ability to perform this behavior was believed to occur due to damage of lateral line hair cells (Buck L M, et al., Hear Res. 2012 February; 284(1-2):67-81; Suli A, et al., PLoS One. 2012; 7(2):e29727). Rheotaxis was defined as fish oriented headfirst, with an axial alignment within 30 degrees on either side of the directional flow vector and attempting to swim into the current. A fish not performing rheotaxis was defined as one oriented head first in a direction greater than 30 degrees from the directional flow vector and/or not swimming at all (FIG. 5). The latter was deemed non-rheotaxis behavior because damaged hair cells prevent sensation of water flow causing abnormal orientation and insufficient stimulus to swim. After raw data acquisition, still images were taken every 5 seconds using a macro designed on ImageJ software. From the still images, the angle of every fish was measured using ImageJ and placed into an Excel spreadsheet for data processing (FIG. 6). The angular data ranged in values from 0-180 degrees and represented an absolute divergence from the directional flow vector. Therefore, there were no negative values or angles measured between 181-360 degrees.

### Drug Exposure—Cisplatin Dose Dependent Exposure

It is known that cisplatin causes hair cell/neuromast damage in a dose dependent manner (Ou H C, et al., Hear Res. 2007 November; 233(1-2):46-53). In order to establish the sensitivity of the newly developed behavior assay for detecting damage caused by varying doses of cisplatin, a cisplatin dose-dependent exposure experiment was prepared. Cisplatin solutions were prepared from powder (Sigma; P4394) in embryo medium. Five dpf larvae were incubated in cisplatin at concentrations of 0, 250, 500, 750, or 1000 μM (Cis 0, Cis 250, Cis 500, Cis 750, and Cis 1000, respectively) for 4 hours at room temperature or 28±0.5° C. then rinsed 3 times in embryo media.

### Drug Exposure—Dexamethasone Pre-Exposure

Intratympanic administration of dexamethasone has been used clinically for treatment of sudden hearing loss, including cisplatin-induced hearing loss (Marshak T, et al., Otolaryngol Head Neck Surg. 2014 Mar. 11; 150(6):983-90; Hill G W, et al., Otol Neurotol. 2008 October; 29(7):1005-11.). In order to assess the effect of dexamethasone on cisplatin-induced ototoxicity using the behavior/anatomical assay technique, dexamethasone pre-exposure groups were prepared. Dexamethasone (Sigma; D4902) stock solution (25 mg/ml) was prepared with DMSO (Dimethyl Sulphoxide, Sigma; D2650) and diluted with embryo medium. Four-and-half dpf larvae were incubated in dexamethasone at a concentration of 5 μM for 12 hours at 28.5±0.5° C. and subsequently rinsed in embryo media. Four-and-half dpf fish were used to allow for a half-day of maturation to ensure all fish would be 5 dpf at time of behavioral/anatomical testing. The dexamethasone pre-treatment was subsequently incubated in 1000 μM cisplatin (Dex 5/Cis 1000) for 4 hours at 28.5±0.5° C. and rinsed following the previously stated protocol. A dexamethasone-alone (Dex 5/Cis 0) group was also created. This group was not exposed to cisplatin after the dexamethasone pre-exposure. In order to eliminate the possibility that the solvent causes any modifications to the hair cells during pre-exposure, DMSO exposure groups (DMSO/Cis 1000 and DMSO/Cis 0) were used for the anatomical assay. DMSO was diluted with embryo medium to the same final concentration (<0.01%) as the dexamethasone solution. DMSO groups were treated with the same protocol of dexamethasone pre-exposure group.

### Results

### Anatomical Assay—Cisplatin Dose-Dependent Exposure

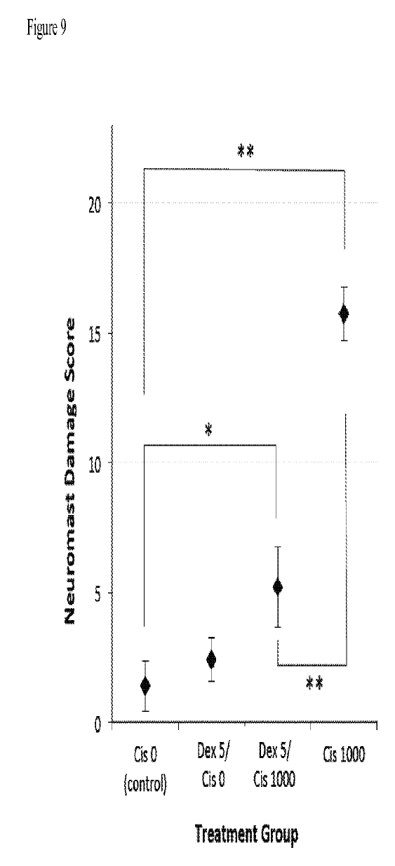

To evaluate damage to neuromasts resulting from exposure to varying concentrations of cisplatin (0, 250, 500, 750, 1000 μM), each neuromast was scored to obtain the total damage score per fish in each condition. As outlined above, the mean score of three evaluators was utilized to determine the final “neuromast damage score,” with the inter-scorer variation validated using the Pearson correlation coefficient (Pearson's r_{AB}=0.97, r_{AC}=0.97, r_{BC}=0.96). Using this approach, the neuromast damage scores revealed a linear, dose-dependent relationship between ototoxic, exogenous agent exposure and increased neuromast damage (Table I, FIG. 7).

### Anatomical Assay—Dexamethasone Pre-Treatment

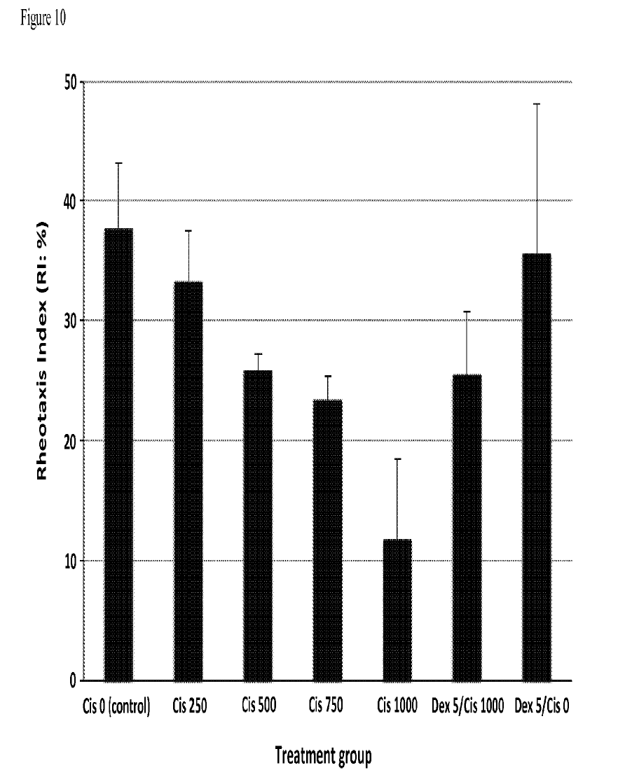

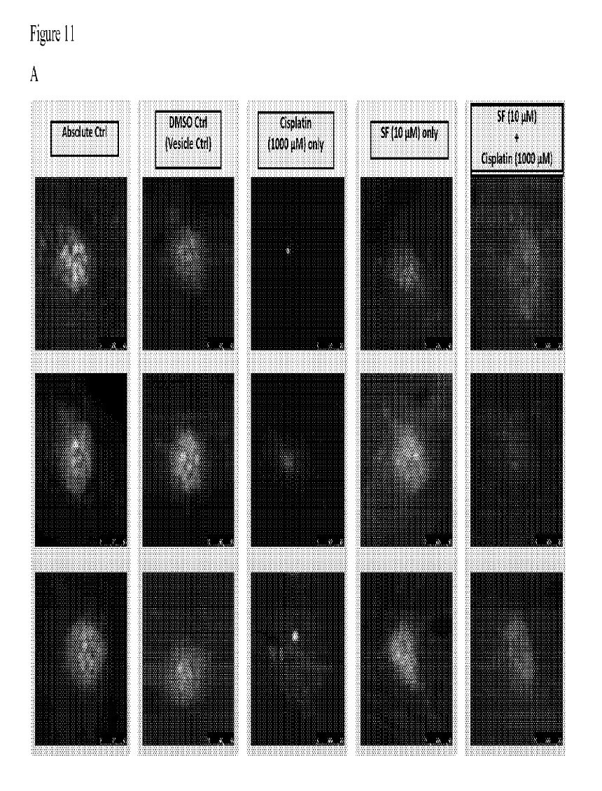

Two different dexamethasone experimental groups: dexamethasone 5 μM/cisplatin 0 μM (Dex 5/Cis 0) and dexamethasone 5 μM/cisplatin 1000 μM exposure (Dex5/Cis 1000) were used. Also, two DMSO groups were prepared: DMSO/cisplatin 1000 μM (DMSO/Cis 1000) and DMSO/cisplatin 0 μM exposure (DMSO/Cis 0). To evaluate otoprotective effects from exposure to varying combinations of dexamethasone and cisplatin, each neuromast was scored and the total damage score per fish in each condition was obtained. Inter-scorer variation was validated using the Pearson correlation coefficient (Pearson's r_{AB}=0.98, r_{AC}=0.97, r_{BC}=0.98). Using this approach no hair cell damage caused by dexamethasone, and partial rescue of hair cell damage against cisplatin (Table I, FIG. 8, FIG. 9) were observed.

### Anatomical Assay—Statistical Analysis

A clear correlation between cisplatin concentration and neuromast damage score was observed in the cisplatin dose dependent experiment. Scores at each level of cisplatin exposure were noted to all be statistically different (one-way ANOVA, p<0.0001). In order to analyze the dexamethasone effect against cisplatin-induced hair cell damage, statistical significance for the neuromast damage scores was evaluated using a t-test. There is no statistically significant difference between Cis 0 and Dex 5/Cis 0. There were statistically significant differences between Cis 1000 and Dex 5/Cis 1000 (p<0.0001) and this result indicates dexamethasone pre-exposure protects/rescues the hair cells from cisplatin-induced damage. However, a statistically significant difference between Cis 0 and Dex 5/Cis 1000 (p<0.001) was found, indicating that pre-exposure with dexamethasone (Dex 5/Cis 1000) “partially” protects hair cells from ototoxic damage. Neither dexamethasone-alone or DMSO-alone (DMSO/Cis 0, (n=3): 2.10±0.39) caused any damage to the zebrafish neuromasts (no statistically significant difference), and DMSO alone didn't show any protective effect against cisplatin (DMSO/Cis 1000, (n=3): 18.22±1.90). No significant differences were found between Cis 1000 vs DMSO/Cis 1000.

### Behavioral Assay Results—Cisplatin Dose-Dependent Exposure

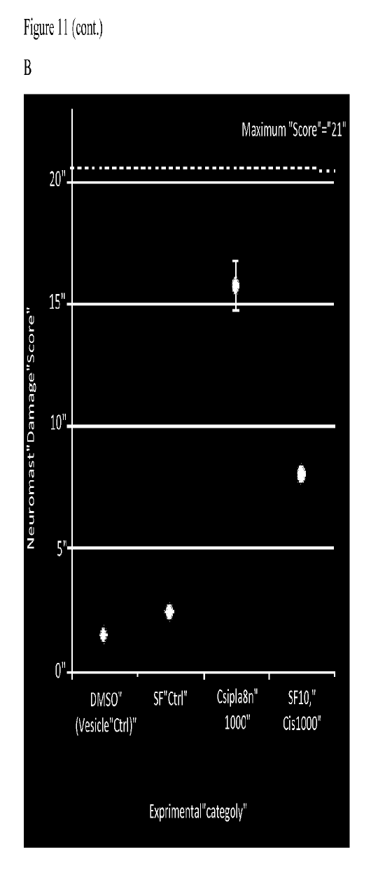

A total of 4515 angles were recorded across all measurement conditions using a semi-automated format that allowed calculation of rheotaxis indices and standard deviations for each treatment group. A clear linear correlation was found between increasing cisplatin dosage and decreased rheotaxis performance, which is consistent with results from the anatomical assay (Table I, FIG. 10).

### Behavioral Assay Results—Dexamethasone Pre-Exposure

A total of 802 angles were recorded across all measurement conditions using a semi-automated format allowing for calculation of rheotaxis indices and standard deviations for each treatment group. Pre-exposure to dexamethasone prior to cisplatin administration significantly improved the ability of fish to perform rheotaxis (Table I, FIG. 10).

### Behavioral Assay Results—Statistical Analysis

Analysis on the entire population was conducted first using Pearson's X². This showed significant differences among the data: X² (6, N=5317)=141.064, p<0.001. Individual Pearson's X² analyses were performed on all possible side-by-side combinations of treatments (Table II). Significant differences were found between Cis 250 vs Cis 500 (p<0.01), and Cis 750 vs Cis 1000 (p<0.001). No significant differences were found between control vs Cis 250 (p>0.05), and Cis 500 vs Cis 750 (p>0.05). These results showed a strong linear correlation between increasing cisplatin dosage and decreased rheotaxis performance. There were also significant differences between Cis 1000 vs Dex 5/Cis 1000 (p<0.001) and Dex 5/Cis 1000 vs Dex 5/Cis 0 (p<0.001). No significant difference was found between Cis 0 vs Dex 5/Cis 0 (p>0.05). These results demonstrate a statistically significant rescue/protective effect with pre-treatment using dexamethasone 5 μM. No damage was induced by dexamethasone-alone treatment.

TABLE 1
Comparison of behavioral and anatomical data between treatment groups

| | Behavioral Assay | Anatomical Assay | |
|---|---|---|---|
| | Rheotaxia Index (%) | Damage Score | |
| Treatment Group | (Mean ± S.D.) | (Mean ± S.D.) | |
| Cis 0 (control) | 37.7 ± 5.5 | 1.36 ± 0.64 | (n = 7) |
| Cis 250 | 33.3 ± 4.3 | 5.40 ± 1.32 | (n = 5) |
| Cis 500 | 25.9 ± 1.3 | 6.87 ± 1.91 | (n = 5) |
| Cis 750 | 23.4 ± 2.1 | 10.40 ± 2.34 | (n = 5) |
| Cis 1000 | 11.6 ± 6.6 | 15.73 ± 1.01 | (n = 5) |
| Dex 5/Cis 1000 | 25.5 ± 5.2 | 5.20 ± 1.53 | (n = 3) |
| Dex 5/Cis 0 | 35.6 ± 12.5 | 2.40 ± 0.64 | (n = 3) |

Abbreviations:
Cis, cisplatin (following number indicates concentrations in μm);
Dex, dexamethasone (following number indicates concentrations in μm);
S.D., standard deviation

TABLE II
Behavioral data statistical analysis Pearson's X²

| | Treatment Group | | | | | | |
|---|---|---|---|---|---|---|---|
| | | | | | | Dex5/ | Dex5/ |
| | Cis0 | Cis 250 | Cis 500 | Cis 750 | Cis 1000 | Cis 1000 | Cis 0 |
| Cis 0 | | 0.06 | 27.82** | 39.93** | 26.29** | 26.43** | 0.727 |
| Cis 250 | | | 11.47* | 20.19** | 71.21** | 11.32* | 0.967 |
| Cis 500 | | | | 1.48 | 35.8** | 0.03 | 27.82** |
| Cis 750 | | | | | 25.24** | 0.975 | 28.64** |
| Cis 1000 | | | | | | 32.67** | 82.22** |
| Dex 5/Cis 1000 | | | | | | | 17.85** |
| Dex 5/Cis 0 | | | | | | | |

*p < 0.01
**p < 0.001
Abbreviations: Cis, cisplain (following number indicates concentrations in μM); Dex: dexamethane (following number indicates concentrations in μM)

### Example 2

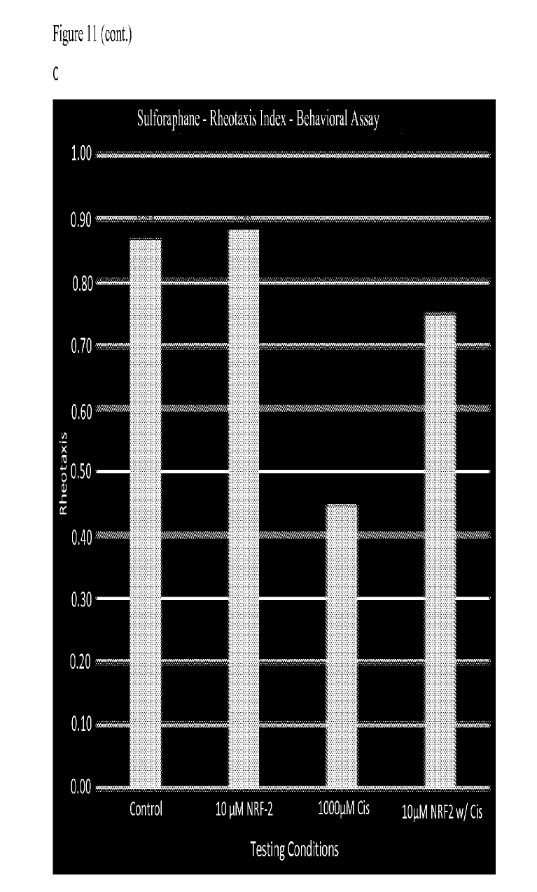

This example demonstrates that small molecule activation of the NRF2 pathway protects zebrafish hair cell anatomy and function. Sulforaphane, a naturally occurring molecule within the isothiocyanate group of organosulfur compounds, is present within cruciferous vegetables such as broccoli. Low doses of SF induce production of antioxidant enzymes such as glutathione transferases, UDP-glucuronyltransferase, NAD(P)H:quinone oxidoreductase I and heme oxygenase-1 (HO-1) via NRF2 mediated activation of ARE promoters upstream from their genes—thereby allowing a diverse array of electrophilic and oxidative toxicants to be eliminated in a coordinated manner. Four-and-half dpf larvae were incubated in sulforaphane at a concentration of 10 μM for 12 hours and subsequently incubated for 4 hours in 1000 μM cisplatin (SF10/Cis 1000). An SF-alone (SF10/Cis 0) group and DMSO vehicle controls (DMSO/Cis 1000; DMSO/Cis 0) were also tested. To assess the otoprotective effects of SF on hair cell anatomy, aggregate neuromast damages were determined for each treatment group. Results found that sulforaphane alone caused no hair cell damage while partial rescue of hair cell architecture was noted for those fish pretreated with SF and then exposed to cisplatin (FIG. 11A/11B).

Then, to determine whether rheotaxis behavior could serve as a biomarker for the observed anatomic protection, swimming angles were recorded across all treatment conditions using next generation video imaging and semi-automated image analysis (detailed calculations of rheotaxis indices). These new image analysis techniques expanded the spread in rheotaxis index between controls (>85%) and damaged 1000 μM fish (45%). Exposure to SF prior to cisplatin administration dramatically improved the ability of fish to perform rheotaxis (FIG. 11C), indicating that improvements in swimming behavior were again predictive of anatomic zebrafish hair cell protection.

All publications and patents mentioned in the above specification are herein incorporated by reference as if expressly set forth herein. Various modifications and variations of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in relevant fields are intended to be within the scope of the invention.

1-10. (canceled)

11. A method of screening compounds for otoprotective or regenerative activity, comprising:
a) contacting a fish that exhibits altered behavior in response to loss of hair cell mass with a ototoxic agent and a test compounds; and
b) identifying test compounds that alter, treat, or prevent ototoxicity caused by said ototoxic agent or have regenerative activity by measuring changes in said behavior.

12. The method of claim 11, wherein said behavior is rheotaxis.

13. The method of claim 11, wherein said fish is Danio rerio.

14. The method of claim 11, wherein said test compounds are pharmaceutical agents or candidate pharmaceutical agents.

15. The method of claim 11, wherein said test compounds are contacted with said fish before, during, or after contact with said ototoxic agent.

16. The method of claim 11, wherein said ototoxic agent is selected from cisplatin, carboplatin, aminoglycoside antibiotics, diuretics, salicylates, NSAIDs, quinine, solvents, and heavy metals.

17. The method of claim 11, wherein a plurality of distinct test compounds are each contacted with different fish or populations of fish.

18. (canceled)

19. The method of claim 11, wherein said behavior is quantitated.

20. The method of claim 19, wherein said quantitation is automated or manual.

21. A system, comprising:
a) a multichamber device comprising a plurality of wells, each well comprising one or more fish or organisms that exhibit altered behavior in response to exogenous agent exposure;
b) one or more fluid exchange components;
c) a detection of behavior component; and
d) a data analysis component.

22. The system of claim 21, further comprising one or more test compounds.

23. The system of claim 21, wherein said fish is Danio rerio.

24. The system of claim 21, wherein said test compound is selected from a candidate otoprotective agent, a candidate ototoxic agent, and a candidate hearing loss treatment.

25. The system of claim 21, wherein said data analysis component calculates hair cell mass loss from said altered behavior.

26. The system of claim 21, wherein said data analysis component comprises one or more of a microscope, a camera, computer processor and computer software.

27. The system of claim 21, wherein said detection of behavior component identifies and quantitates altered behavior of said fish or organism.

28. The system of claim 21, wherein said device comprises at least 10 wells.

29-31. (canceled)

32. A method of treating or preventing hearing loss, comprising:
administering sulforaphane to a subject at risk of hearing loss or demonstrating symptoms of hearing loss.

33. The method of claim 32, wherein said sulforaphane alters, treats, or prevents ototoxicity caused by an ototoxic agent or has regenerative activity on hair cell loss.

34. The method of claim 33, said ototoxic agent is selected from cisplatin, carboplatin, aminoglycoside antibiotics, diuretics, salicylates, NSAIDs, quinine, solvents, and heavy metals.

35-37. (canceled)
